(19) United States
(12) Patent Application Publication
(10) Pub. No.: US 2026/0024611 A1
(43) Pub. Date: Jan. 22, 2026

(54) METHOD FOR PREDICTING AND SCREENING INTERACTIONS BETWEEN LACTOBACILLUS BULGARICUS AND STREPTOCOCCUS THERMOPHILUS TWO-BY-TWO

(71) Applicant: Inner Mongolia Agricultural University, Hothot (CN)

(72) Inventors: Zhihong SUN, Hothot (CN); Gaifang DONG, Hothot (CN); Mei BAI, Hothot (CN); Jie YU, Hothot (CN); Jie ZHAO, Hothot (CN); Weicheng LI, Hothot (CN); Hao JIN, Hothot (CN)

(73) Assignee: Inner Mongolia Agricultural University, Hothot (CN)

(21) Appl. No.: 19/346,555

(22) Filed: Sep. 30, 2025

(30) Foreign Application Priority Data
Jan. 8, 2025 (CN) 202510029908.3

(51) Int. Cl. G16B 5/00; C12Q 1/02; G16B 20/00

(52) U.S. Cl. CPC G16B 5/00; C12Q 1/025; G16B 20/00

(57) ABSTRACT

A method for predicting and screening symbiotic interactions between Lactobacillus bulgaricus and Streptococcus thermophilus is provided, belonging to the technical field of fermented milk production. In the method, a comprehensive feature vector is generated by combining KEGG features and k-mer feature frequencies of Lactobacillus bulgaricus and Streptococcus thermophilus strains. The top 200 important features are screened from the real labeled samples using the chi-square test, gradient boosting, and variance analysis. Subsequently, pseudo-labeled samples are generated using GAN, and a machine learning model is constructed by combining the real labeled samples, which is configured to predict the interaction effects of strain combinations. Finally, the accuracy of the model predictions is verified through fermentation experiments, and the optimal model is selected. The present disclosure can efficiently predict the potential for symbiotic interaction between strains, thereby improving the efficiency and quality of fermented milk production.

### TECHNICAL FIELD

The present disclosure relates to the technical field of food production, specifically to the technical field of fermented milk production, and particularly to a method for predicting and screening symbiotic interactions between Lactobacillus bulgaricus and Streptococcus thermophilus.

### BACKGROUND

Fermented milk is a curd-like product made by fermentation of Lactobacillus bulgaricus and Streptococcus thermophilus in milk (sterilized milk or concentrated milk) with or without milk powder (or skim milk powder). The finished product contains a large number of corresponding active microorganisms. Fermented milk is characterized by its high nutrient content, including calcium, protein, riboflavin, and vitamins. Currently, it has been proven that fermented milk has the effects of balancing intestinal flora, improving immunity, lowering cholesterol and delaying aging. An increasing number of people consume fermented milk on a daily basis, raising more stringent requirements for the quality of fermented milk production.

In the prior art, two strains of Streptococcus thermophilus and two strains of Lactobacillus bulgaricus are put together, randomly combined through biological experiments, the phenotypic data of acid production rate and proteolysis ability is test, and finally determines whether the four strains can interact symbiotically to accelerate the fermentation speed in the fermented milk production process, and improve the fermentation properties such as viscosity and water holding capacity. This method is time-consuming and labor-intensive, and yields low output. And the process of determining whether a group of bacteria interacts may be taken 3-4 months.

### SUMMARY

An objective of the present disclosure is to provide a method for predicting and screening interactions between Lactobacillus bulgaricus and Streptococcus thermophilus two-by-two, which can achieve high-throughput and efficient prediction while guaranteeing prediction accuracy.

In order to achieve the above objective, the present disclosure provides a method for predicting and screening interactions between Lactobacillus bulgaricus and Streptococcus thermophilus two-by-two, and the method includes the following steps:
step S1, calculating k-mer data of a whole genome of two strains of Lactobacillus bulgaricus and two strains of Streptococcus thermophilus, respectively, calculating respective Σ4^{k} dimensional feature vectors according to the k-mer data, and forming a Kyoto Encyclopedia of Genes and Genomes (KEGG) matrix by calculating a gene copy number of each strain;
step S2, fusing the KEGG features of the four strains according to a principle of adding copy numbers of overlapping genes and replicating copy numbers of non-overlapping genes, thereby obtaining n features; obtaining m features by accumulating the k-mer feature frequencies of the four strains; and obtaining n+m features by concatenating n features and m features;
step S3, setting a number of real labeled samples to p, and screening a top 200 features in a feature importance ranking list by three feature selection methods of a chi-square test, a gradient boosting and a variance analysis on n+m features according to the p real labeled samples;
step S4, for p real labeled samples, completing an iterative process of generating false data and discriminating true and false data by alternately working with a generator and a discriminator of generative adversarial networks (GAN), and finally generating 10×p pseudo labeled samples;
step S5, constructing a machine learning model based on the real labeled sample and the pseudo labeled sample, and then predicting Lactobacillus bulgaricus and Streptococcus thermophilus based on a two-by-two combination by using the constructed machine learning model; and
step S6, performing fermentation experiments by selecting multiple combinations from predicted results, comprehensively evaluating fermentation effect of the strain combination according to the fermentation features, and selecting an optimal model with a highest prediction accuracy by comparing the experimental results with the prediction results of the machine learning model.

In some embodiments, in step S1, k=5-9.

In some embodiments, in step S5, the machine learning model includes logistic regression (LR), support vector machine (SVM), random forest (RF), K-nearest neighbor (KNN), and Gaussian naive Bayes (GNB).

Therefore, the present disclosure adopts the above-mentioned method for predicting and screening interactions between Lactobacillus bulgaricus and Streptococcus thermophilus two-by-two, and the beneficial technical effects are as follows:

A high-precision prediction model for the interaction between two strains of Lactobacillus bulgaricus and two strains of Streptococcus thermophilus is successfully constructed through in-depth analysis of the genomes of Lactobacillus bulgaricus and Streptococcus thermophilus, combining with a series of operations such as KEGG operation, k-mer feature extraction, fine feature selection, and GAN data enhancement. This model can efficiently predict whether any combination of these four strains can achieve interaction and symbiosis in batches.

In the feature selection process, the feature combinations that have the most significant impact on the interaction are accurately screened out, thus ensuring that the prediction model can focus on the most critical information. Meanwhile, the efficiency of machine learning modeling is further improved with the help of data enhancement technology, which not only improves prediction efficiency and throughput, but also ensures the accuracy of prediction results. The implementation of these optimization measures collectively promotes the potential application of the present disclosure in the dairy fermentation and other related fields.

### DETAILED DESCRIPTION OF THE EMBODIMENTS

The technical scheme of the present disclosure is further explained below by embodiments.

Unless otherwise defined, the technical or scientific terms used in the present disclosure shall be those to which the present disclosure belongs.

### Embodiment 1

A method for predicting and screening interactions between Lactobacillus bulgaricus and Streptococcus thermophilus two-by-two, the method includes the following steps:
step S1, feature extraction of Lactobacillus bulgaricus and Streptococcus thermophilus.

The k-mer (k=5-9) data of the whole genome of two strains of Lactobacillus bulgaricus and two strains of Streptococcus thermophilus are calculated, respectively.

The respective Σ4^{k} dimensional feature vectors are calculated according to the k-mer data, and the gene copy number of each strain is calculated by CENSOR, CNVnator, and other software to form the KEGG matrix.
Step S2, feature combination of two strains of Lactobacillus bulgaricus and two strains of Streptococcus thermophilus.

The KEGG features of the four strains are fused according to the principle of adding copy numbers of overlapping genes and replicating copy numbers of non-overlapping genes, and n features are obtained.

m features are obtained by accumulating the k-mer feature frequencies of the four strains.

n+m features are obtained by concatenating n features and m features.
Step S3, feature selection is performed according to a small amount of existing labeled data.

If the number of labeled samples is p, the top 200 features are screened in a feature importance ranking list of the three methods by three feature selection methods of the chi-square test, the gradient boosting and the variance analysis on n+m features according to the p labeled samples;
Step S4: data enhancement.

For p labeled samples, the iterative process of generating false data and discriminating true and false data is completed by alternating steps with the generator and discriminator of GAN, and finally 10×p pseudo labeled data is generated.
Step S5: Model construction.

Five machine learning models are constructed using 11×p samples (10p are generated samples, and p is real label positive sample) with LR, SVM, RF, KNN and GNB modeling. The models are used to predict 265,364, 100 2:2 combinations composed of 181 strains of Lactobacillus bulgaricus and 181 strains of Streptococcus thermophilus existing in the laboratory, thereby obtaining the model prediction results of all combinations (0 or 1, 0 denotes no interaction, 1 denote interaction), and the prediction results are submitted to the laboratory for verification.
Step S6, laboratory verification and determination of optimal model.

30 groups are randomly selected from step S5 to perform fermentation experiments, and then 30 groups of strain combinations are comprehensively evaluated to determine whether the fermentation labels are 0 or 1 based on the fermentation features such as fermentation time, viscosity, and water holding capacity obtained from the fermentation experiments.

The results of laboratory verification are compared with the prediction results of five machine learning models, and the optimal model is selected, which is the logistic regression model.
Step S7, a set of experiments is performed by using the optimal model obtained in step S6, that is, the interaction between two strains of Lactobacillus bulgaricus (IMAU20360 and IMAU20428) and two strains of Streptococcus thermophilus (IMAU10630 and IMAU40145) is predicted. The prediction result is output in five seconds, and the prediction results indicated interaction. It shows that the time consumed in screening a 2:2 starter strain according to the present disclosure is much less than the time consumed in the laboratory.

It should be noted that any content not detailed in the present disclosure is prior art and is well known to those skilled in the art.

Therefore, the present disclosure uses the above-mentioned method for predicting and screening interactions between Lactobacillus bulgaricus and Streptococcus thermophilus two-by-two, which can achieve high-throughput and efficient prediction while guaranteeing prediction accuracy.

Finally, it should be noted that the above embodiments are merely used for describing the technical solutions of the present disclosure, rather than limiting the same. Although the present disclosure has been described in detail with reference to the preferred examples, those of ordinary skill in the art should understand that the technical solutions of the present disclosure may still be modified or equivalently replaced. However, these modifications or substitutions should not make the modified technical solutions deviate from the spirit and scope of the technical solutions of the present disclosure.

What is claimed is:

1. A method for predicting and screening symbiotic interactions between Lactobacillus bulgaricus and Streptococcus thermophilus, comprising the following steps:
step S1, calculating k-mer data of a whole genome of two strains of Lactobacillus bulgaricus and two strains of Streptococcus thermophilus, respectively, calculating respective Σ4^{k} dimensional feature vectors according to the k-mer data, and forming a KEGG matrix by calculating a gene copy number of each strain;
step S2, fusing the KEGG features of the four strains by adding copy numbers of overlapping genes and replicating copy numbers of non-overlapping genes, thereby obtaining n features;
obtaining m features by accumulating the k-mer feature frequencies of the four strains; and
obtaining n+m features by concatenating n features and m features;
step S3, setting a number of real labeled samples to p, and screening a top 200 features in a feature importance ranking list according to three feature selection methods:
a chi-square test;
gradient boosting; and
a variance analysis of n+m features according to the p real labeled samples;
step S4, for p real labeled samples, completing an iterative process of generating false data and discriminating true and false data by alternately working with a generator and a discriminator of GAN, wherein 10×p pseudo labeled samples are generated;
step S5, constructing a machine learning model based on the real labeled sample and the pseudo labeled sample, and then predicting symbiotic interactions between Lactobacillus bulgaricus and Streptococcus thermophilus using the constructed machine learning model; and
step S6, performing fermentation experiments by selecting a plurality of combinations from predicted results, comprehensively evaluating fermentation effect of the strain combination according to the fermentation features, and selecting an optimal model with a highest prediction accuracy by comparing the experimental results with the prediction results of the machine learning model.

2. The method for predicting and screening symbiotic interactions between Lactobacillus bulgaricus and Streptococcus thermophilus according to claim 1, wherein in step S1, k=5-9.

3. The method for predicting and screening symbiotic interactions between Lactobacillus bulgaricus and Streptococcus thermophilus according to claim 1, wherein in step S5, the machine learning model comprises logistic regression, support vector machine, random forest, K-nearest neighbor, and Gaussian naive Bayes.
